(19) United States
(12) Patent Application Publication
(10) Pub. No.: US 2012/0095502 A1
(43) Pub. Date: Apr. 19, 2012

(54) WOUND CLOSURE DEVICE

(75) Inventors: Rainer Bargon, Tuttlingen (DE); Erich Odermatt, Schaffhausen (CH); Bernd Stöver, Schenefeld (DE)

(73) Assignee: AESCULAP AG, Tuttlingen (DE)

(21) Appl. No.: 13/266,825

(22) PCT Filed: Nov. 11, 2009
(86) PCT No.: PCT/EP09/08029

§ 371 Date: Dec. 12, 2011

(30) Foreign Application Priority Data
Apr. 29, 2009 (DE) 10 2009 020 763.5

(51) Int. Cl. A61B 17/03

(52) U.S. Cl. 606/216

(57) ABSTRACT

An adjustable wound closure device includes at least one band or strip retaining element provided for attachment in the region of at least one edge of a wound. The retaining element has a multiplicity of hook elements on at least part of at least one of its two faces. The wound closure device further includes at least one gauze cover element which substantially completely covers the wound and the edge regions provided with the retaining element. The hook-like elements of the retaining element and the meshes of the cover element are designed and arranged such that the wound can be reversibly closed by a positive engagement of at least some of the hook-like elements in the meshes.

### RELATED APPLICATIONS

This is a §371 of International Application No. PCT/EP2009/008029, with an international filing date of Nov. 11, 2009 (WO 2010/124712 A1, published Nov. 4, 2010), which is based on German Patent Application No. 10 2009 020 763.5, filed Apr. 29, 2009, the subject matter of which is incorporated by reference.

### TECHNICAL FIELD

This disclosure relates to an adjustable wound closure device for closing wounds and to a kit containing such a wound closure device.

### BACKGROUND

A wound generally describes the separation of tissue on external or internal surfaces of the body. Such wounds in humans or animals can be caused by temperature (burns, cold injuries) or by chemicals (e.g., acid burns). However, wounds are often caused by mechanical action, and these mechanical wounds not only occur by accident but also have to be necessarily accepted in some cases, for example, in surgery. In the latter case, the wounds are also referred to as incisions, i.e., cuts deliberately made in the human body, for example, during surgical interventions.

In many cases, particularly after surgery, the wounds have to be at least temporarily closed or protected from the environment by using some type of aid. This is primarily done to prevent the entry of foreign materials, in particular of pathogens, and to accelerate the wound-healing process.

Corresponding wound closure devices in a great many designs are of course already known. These range from plasters and dressings to special wound closures for specific uses. Of course, mention must also be made of suturing, in which the wound is closed with the aid of a needle and resorbable or non-resorbable threads.

These and other wound closure devices are generally provided either for closing the skin or for closing wounds within the body, e.g., for fascia closure.

The previously known solutions for wound closure will not be discussed in any great detail. Thus, wound closure devices based on staples, suture material or adhesives have of course been known for decades. The use of adhesive strips/plaster strips is also already documented with a large number of designs. Even comparatively unusual wound closure devices such as screw closures or magnet closures have been discussed and in some cases also put into use.

Suture-free wound closure devices have also already been described, in which anchoring elements are fixed to the wound edge and then tightened relative to one another by tying. In this way, the wound edges are moved toward each other and the wound thereby closed. One such design is disclosed in U.S. Pat. No. 7,429,265.

A disadvantage of this design is that a large number of anchoring elements (retaining elements) have to be applied to the wound edge to close the wound along its entire length with a substantially constant tension. Moreover, in US '265, the retaining elements are anchored in the tissue with the aid of prongs, which leads to additional injury of the tissue. Finally, that design means that applying the wound closure device to the wound is time-consuming.

Moreover, wound closure devices are also already known that are based on hook-and-loop fasteners or use hook-and-loop fasteners. In this connection, reference may be made to U.S. Pat. No. 5,876,365, U.S. Pat. No. 7,414,168 and U.S. Pat. No. 4,825,866.

U.S. Pat. No. 5,876,365 describes an adhesive bandage with an opening which is delimited all the way round by the bandage and through which the wound remains accessible from above. For reversible closure of the opening, a planar and closed cover element is provided, which can be secured to the edge of the opening with the aid of hook-and-loop strips.

U.S. Pat. No. 7,414,168 describes a two-component hook-and-loop closure system for use in lacerations or incisions, which system consists of two plasters with an adhesive underside. The two plasters are applied to the opposite sides of a wound. They have laterally arranged strips which, across the wound, can be secured with the aid of a hook-and-loop fastener to the surface of the opposite plaster. In this way, the wound edges are drawn toward each other and the wound is closed.

In U.S. Pat. No. 4,825,866, adhesive strips are likewise arranged in the manner of plasters on opposite sides of the wound, but in that case they are drawn toward each other and fixed with separate parallel strip elements.

Finally, reference is also made to DE 20 2006 015 861 U1, which likewise discloses a wound closure device based on the principle of the hook-and-loop fastener. Two hook strips coated on their underside with an adhesive film are likewise affixed to the skin on both sides parallel to the wound edges. The wound edges are fixed, and the wound thus closed, with the aid of individual dumbbell-shaped adhesive bodies, which can be secured on the two hook strips.

It can be stated in summary that, because of the disadvantages of the previously known wound closure devices, there is still a need for new wound closure devices, particularly ones that can be (subsequently) adjusted. There is a need for such wound closure devices to, for example, avoid a compartment syndrome after fractures of the lower arm or lower leg. A suitable wound closure device should prevent damage to the nerves as a result of compression within the fascias, which can often be triggered by bacterial inflammation, particularly after accidents, and contamination of the wound. Moreover, wound closure using suture material poses the risk of needlestick injuries and associated scar formation. Considerable quantities of suture material are needed for larger wounds. Finally, the risk of personnel being infected by the patient's blood must be mentioned, as well as the aforementioned risk of compartment syndrome developing as a result of infection of the patient.

Because of the metal material used, stapling systems are often unsuitable for magnetic resonance and computed tomography. Moreover, the risk of scar formation caused by the staples is considerable.

Adhesives, insofar as they can be used at all in view of the comparatively low tensile strength of the wound closure, are not very suitable for weeping wounds. This also applies to wound plasters, which in general can only be used topically.

In the reversible closure techniques, in particular in the examples mentioned, a distinction can generally be made between two variants. On the one hand, there are designs that are comparatively easy and quick to use but that are generally limited to straight wound profiles. On the other hand, there are wound closure devices that can also be used for more complex wound profiles but that are generally time-consuming and comparatively complicated to use.

Accordingly, it could be helpful to make available a novel wound closure device or novel wound closure system that greatly reduces the stated disadvantages. Thus, it is designed to allow the user, i.e., generally the physician or the operating surgeon, to close a wound relatively quickly. For this purpose, the user is to be provided with a system that is easy to follow and easy to handle. However, the novel wound closure device should be suitable not only for simple wound profiles, i.e., generally straight wound profiles, but also for more complicated wound profiles, for example, oblique or curving wound profiles. Finally, the materials and components used for the wound closure device should be as inexpensive as possible to keep the overall costs as low as possible. Ideally, it should be possible here to use materials and components that are already known.

### SUMMARY

We provide an adjustable wound closure device including at least one band or strip retaining element that attaches in a region of at least one edge of a wound, wherein the retaining element has a multiplicity of hook elements on at least part of at least one of its two faces, and at least one gauze cover element that substantially completely covers the wound and the edge regions provided with the retaining element, wherein the hook elements of the retaining element and the meshes of the cover element are arranged such that the wound can be reversibly closed by a positive engagement of at least some of the hook elements in the meshes.

We also provide a kit for closing wounds including at least one wound closure device in sterilized form in a sterile package.

We further provide a method for closing wounds, wherein at least one band or strip retaining element, which has a multiplicity of hook elements on at least part of at least one of its two faces, is applied in a region of at least one edge of a wound, and at least one gauze cover element, that substantially completely covers the wound and the edge regions provided with the retaining element is arranged over the wound and the retaining element such that wound closure takes place through positive engagement of elements of the retaining element in meshes of the cover element.

### BRIEF DESCRIPTION OF THE DRAWINGS

FIG. 1 shows a first example of the wound closure device in a schematic view (plan view).

FIG. 2 shows another example of the wound closure device in a schematic view (plan view).

FIG. 3 shows a third example of the wound closure device in a schematic view (cross section).

FIG. 4 shows a special example of a retaining element with liquid-repelling means.

FIGS. 5a,b shows a schematic view of a wound being closed with the aid of a wound closure device.

### DETAILED DESCRIPTION

Our wound closure device is (subsequently) adjustable or adaptable and is provided for closing wounds. It has, on the one hand, at least one band-like or strip-like retaining element provided for attachment in the region of at least one edge of a wound. The retaining element has a multiplicity of hook-like elements on at least part of at least one of its two faces. On the other hand, the wound closure device comprises at least one gauze-like cover element intended to substantially completely cover the wound and the edge regions provided with the retaining element.

The hook-like elements of the retaining element and the meshes of the cover element are designed and/or arranged such that the wound can be reversibly closed by a positive engagement of at least some of the hook-like elements in the meshes.

Reversibly means that hook-like elements and meshes are separated from each other again without damage and the processes of closing and opening can be repeated many times. To this extent, the wound closure device can also be referred to as a “reversible” wound closure device.

“Wound closure device” is to be understood here as any device with which the wound edges are at least partially approximated to each other (adapted) and with which the wound is closed in the manner described. This distinguishes our wound closure device from other types of wound closure such as suturing, stapling or adhesion. “Adjustable” is to be understood as meaning that the wound closure device, after it has first been applied, can be changed during the period of the wound-healing process, i.e., can be adapted to the particular state of wound healing. This is important particularly during the first days after the wound closure device has been applied. Accordingly, after it has first been applied, the wound closure device can be opened again and, if appropriate, reapplied. This permits monitoring of the wound and adaptation of the closure depending on the course of the wound-healing process.

The wound closure device can comprise an additional film, which can be placed over the wound closure device after the wound has been closed. This is not therefore a film connected beforehand to the gauze-like cover element, but instead a film that can be arranged subsequently over the wound closure device and thus at least over the cover element.

This additional film serves to close the wound areas still exposed through the mesh structure of the gauze-like cover element. The film can preferably be a plastic film, in particular, a film of polyurethane. Polyurethane and, if appropriate, other suitable plastic materials are permeable to gas and to water vapor, such that a suitable exchange of substances is permitted between wound and environment. Moisture control can thus be adjusted via the thickness and chemical composition of the film, which would otherwise be possible only through use of hydrocolloid particles, for example, as in the registered product Urgotül®, which particles may in rare cases cause allergies.

The additional film that can be placed over the wound closure device can, at edge regions protruding beyond the dimensions of the wound closure device (including the securing areas thereof at the retaining elements) during the intended use of the film, have securing means for fastening to the skin or the other tissue. These can, in particular, be adhesive means such as adhesive strips and the like. This ensures that the coverage of the actual wound closure device by the film is permanently maintained. The securing means, preferably adhesive means, can preferably be placed reversibly on the skin or the tissue, so as to ensure later removal of the film in a simple way.

The retaining element is preferably made of plastic. This is, in particular, a biocompatible plastic. Biocompatible means that the material in question has no negative effect on the organism that is provided with the wound closure device.

A great many different plastics can in principle be used as the plastic materials for the retaining element. Particular mention may be made here of polypropylene (PP), polystyrene (PS), acrylonitrile butadiene styrene (ABS), polyester (PES) or polyamides (PA).

The retaining element can in principle be secured to the wound edge in many different ways, for example, by anchoring means that secure the retaining element to the skin or to the other tissue. However, on the face directed away from the hook-like elements, the retaining element preferably has adhesive means for attaching the retaining element to the wound edge. This is in particular an adhesive layer, preferably one applied across the entire surface in question.

The adhesive means, in particular the adhesive layer, are preferably designed such that the retaining element, after it has been used as intended, can be removed again from the skin or the corresponding tissue. This can be made easier by a tapering shape of the retaining elements.

The adhesives used can be all possible biocompatible adhesive agents, in particular, those that are compatible with skin and/or tissue. Such adhesives, in particular the adhesive layer, preferably consist of alkoxy acrylates or alkyl acrylates or of polymers of butadiene-based or isoprene-based rubbers. Customary copolymers of such adhesives can also be used here.

The length of the band-like or strip-like retaining elements can in principle be chosen freely. Thus, such retaining elements can be made specially available for specific types of wounds and lengths of wounds, for example, in the form of bands or strips of 1 cm long to 20 cm long. However, the retaining elements are preferably made available in the form of quite long bands or strips, for example, also in the form of rolls, from which the suitable retaining element can then be cut off in the desired length.

The width and height of the retaining element (the latter including the hook-like elements) can also be chosen freely. However, widths of less than 20 mm, in particular widths of 5 mm to 10 mm, are preferred. As regards height, values of 0.5 mm to 5 mm are preferred, in particular values of 0.5 mm to 1.5 mm.

As regards the height, and in particular also as regards the width, it should be noted that the retaining element can be made all the more flexible the smaller its height and, in particular, the smaller its width. Narrow and possibly also low retaining elements with a high degree of flexibility can be adapted and applied particularly well to wound profiles that are not straight, for example, oblique or curved wound profiles.

The hook-like elements of the retaining element which then engage in meshes of the gauze-like cover element can in principle be designed in any desired manner. The only condition is the stated hook-like structure, which ensures that the hook-like elements engage behind the threads defining the meshes.

The hook-like elements of the retaining element are preferably shaped like a mushroom head. The threads of the meshes are then therefore secured under the mushroom head on the “hook-like” structure present there.

The hook-like elements of the retaining element can have barbs. These are (mostly additional) hooks, of which the tip is directed counter to the direction of the main hook. This achieves particularly good anchoring and securing.

In particular, such barbs are harpoon-shaped. Such shapes are known in particular from angling.

“Gauze-like” cover element is to be understood as any cover element in which a gauze structure is constructed with the aid of thread-like materials. This gauze structure has an elasticity such that the size of the meshes of the gauze can be (reversibly) increased and/or decreased by tension. This can, in particular, involve knitted fabrics or woven fabrics.

The gauze-like cover element is preferably a woven fabric, in particular, a fabric called “tulle.” The gauze-like structure of tulle is obtained when, during weaving, two warp threads lying together are twisted after each shot. Corresponding woven fabrics are used for net curtains among other things.

It is preferable in principle if the gauze-like cover element is made of plastic threads, in particular, of non-resorbable plastic threads. Such plastic threads can, in particular, consist of polyester, polyamide or polypropylene. Particular mention is also made of the material called “elastane,” a block copolymer of the constituents polyurethane and polyethylene glycol. This material can be mixed (blended) with other materials such as polyamides, polypropylene or polyester.

The gauze-like cover element preferably may have a weight of 10 to 100 g/m². Within this range, weights of 15 to 80 g/m² are preferred.

In particular, the gauze-like cover element has an elongation at break of 30% to 100%, preferably of 30% to 60%. Therefore, the usual stresses that occur during the closure of a wound can generally be taken up without difficulty.

The elongation at break is the material parameter defining the lengthening that has occurred in a sample when the latter breaks, relative to the initially measured length.

Preferably, the gauze-like cover element is designed on at least one of its edges such that at least one planar means, or a means made from individual threads or a plurality of threads, is provided there for orienting the cover element with respect to the wound and with respect to the retaining element. Such means facilitate the orientation and therefore also the adjustability of the wound closure device as a whole.

For example, individual threads, several threads or planar areas, which are preferably narrow, can be provided (“protruding”) on one edge or on several edges of the cover element. With the aid of these means, the cover element can then be oriented and also tensioned with respect to the wound and with respect to the retaining element. In this state defined by the means, the cover element is then secured by engagement of the meshes in the hook-like elements of the retaining element and the wound is closed in the desired manner.

Such orienting means or also tensioning means on the cover element also facilitate a subsequent possible separation of the cover element from the retaining elements since the user can use these means to easily remove, for example, successively the cover element from the retaining means (again). In this way, the wound is again (successively) exposed, and the possibly necessary adjustment/adaptation of the wound closure device to the wound-healing process can be made.

The gauze-like cover element is optionally at least partially provided with a coating or film on at least one of its two faces, i.e., on its top face and/or its underside. In this way, a closed cover can be made available over part of the wound or over the entire wound. In the gauze-like cover element, which substantially completely covers the wound, those parts of the wound that are covered by the open parts of the meshes are in fact open.

The coating or film is preferably not present on those regions of the gauze-like cover element that are provided for cooperation with the hook-like elements of the retaining element. This ensures that the meshes are still open at these locations without coating/film and can thus cooperate with the hook-like elements.

In the devices with coating or film, the mesh structure of the gauze-like cover element is connected to the coating or film such that a uniform cover element results which can be handled as such. Designs with an (initially) separate film have already been described.

The coating or film already described above is preferably permeable to water vapor and, in particular, also permeable to gas. In this way, an exchange of water and gas between wound and environment is possible through the cover element, which is conducive to the healing process.

The coating or film preferably involves coatings or films of plastic. Special mention is made here of polyurethane as a material permeable to gas and permeable to water vapor. Also preferred are polymers or copolymers with polar functional groups such as —OR or —COOR, as are found, for example, in polysaccharides or polyalcohols. R stands for H or alkyl, in particular C₁-C₆ alkyl.

As was mentioned at the outset, the wound closure is in the final analysis obtained through positive engagement of at least some of the hook-like elements of the retaining element(s) in the meshes of the cover element (i.e., in at least some of the meshes). Accordingly, the number, dimensions and/or structure of the hook-like elements of the retaining element, on the one hand, and of the meshes/threads of the cover element, on the other hand, can be adapted to one another in the desired manner. For example, the number of hook elements engaging in one mesh can be determined by the choice of the arrangement and/or dimensions of the hook elements and/or by the choice of the mesh size and/or the elasticity of the gauze.

In this connection, it is preferable if the mesh width of the gauze-like cover element is at least twice as great as the diameter of the hook-like elements of the retaining element. This ensures that the hook-like elements fit into the meshes, and therefore cooperation can easily take place between the threads, which define the meshes, and the hook structure of the retaining elements.

In this connection, it is also preferable if the mesh width of the gauze-like cover element is two to four times as great as the diameter of the hook-like elements of the retaining element.

It is also preferred that the arrangement of the hook-like elements on the retaining element is chosen such that between one and three hook-like elements fit into one mesh of the gauze-like cover element. In this way, it is possible to ensure that often more than one hook-like element engages in one mesh, and therefore a cooperation between thread and hook element does not take place only at a thread boundary of a mesh.

Further preferably, for specific wound shapes and wound profiles, our device ensures that, relative to the arrangement of the retaining element on the wound edge, different numbers of hook-like elements of the retaining element fit into one mesh in the direction of the profile of the wound (“along the wound profile”) and in the direction of the wound tensioning to be applied to the wound (“transverse to the wound profile”) of the gauze-like cover element. In this way, by means of the cooperation of hook-like elements and meshes, a stronger tension can optionally be applied to the wound in one direction than in the other direction. In particular, one hook-like element may fit into one mesh in the horizontal direction and two hook-like elements may fit into one mesh in the vertical direction.

Because of the variable and in this case decreasing mesh width, fewer hook-like elements on average can engage in each mesh upon tensile loading of the cover element. However, the possible resulting loss of hold of the hook elements in the meshes is then compensated by the additional tensioning of the cover element.

In connection with the previous examples, mention is also made of the preferred feature of the wound closure device, namely that the holding strength of the wound closure device on the wound, upon tensile loading of the gauze-like cover element perpendicular to the wound profile, i.e., in the horizontal direction (see above), is at least 10 N (Newton). The tensile loading is preferably 12 N to 20 N.

Preferably, the retaining element has liquid-retaining and/or liquid-repelling means on at least one of its two longitudinally extending edges (i.e., those edges possibly adjoining the wound). With the aid of such means, it is possible to reduce or prevent movement of wound liquid, blood or the like out of the wound into the hook structure of the retaining element.

Such a measure is of great advantage since the hook structure of the retaining element has many hollow spaces in which wound liquid, blood and the like may possibly accumulate and become trapped. This can lead to problems in the wound-healing process, for example, caused by invasion and multiplication of germs.

Such liquid-retaining and/or liquid-repelling means can be configured in many different ways. In particular, they can involve a layer or a coating which, as a kind of barrier layer or barrier coating, reduces or prevents the movement of wound liquid, blood or the like into the hook structure of the retaining element. This layer or coating is then provided (at least) on the edge of the retaining element directly adjacent to the edge of the wound.

The layer or coating can preferably be configured in the form of an edge bead which, by virtue of its shape, additionally performs a sealing function. In particular, it is alternatively or additionally possible to produce the layer or coating from a foam material. As is known, foams are artificially produced materials with a cell structure and low density that can be produced from a large number of plastics. Such foam materials are not only able to impede the liquids possibly emanating from a wound but are also optionally able to absorb these liquids and thus keep them away from the hook structure of the retaining element.

As has already been mentioned, the layers or coatings used as liquid-retaining and/or liquid-repelling means can preferably be made from plastics. In addition to polyurethane and polyethylene, use is preferably made here of the materials that were mentioned for the coating or film in the gauze-like cover element. Reference is made to the corresponding statements.

As foams, it may be preferable to use polyethylene foams, in particular polyethylene foam strips. Such foam strips are a few millimeters wide and up to a few millimeters high and have an absorption capacity for liquids such as water of as much as 5% by volume. Such strips can be secured, in particular affixed, to at least one (longitudinal) edge of the retaining element or to a region of the underside of the retaining element adjacent to this edge.

The wound closure device is preferably present in sterile form. In particular, it is arranged in a sterile package. The sterile package ensures that the user, generally the physician or operating surgeon, only has to remove the wound closure device from the package and can immediately use it.

As has already been mentioned at the outset, we also provide a kit for closing wounds. This kit comprises at least one wound closure device as has been described. This kit comprises the wound closure device in sterilized form, preferably in a sterilized package.

We further provide for the use of a gauze-like element in connection with a band-like or strip-like retaining element which has a multiplicity of hook-like elements on at least part of at least one of its two faces, for substantially complete closure of wounds.

Finally, we provide a method for closing wounds, wherein at least one band-like or strip-like retaining element, which has a multiplicity of hook-like elements on at least part of at least one of its two faces, is applied in the region of at least one edge of a wound, and at least one gauze-like cover element, which is intended to substantially completely cover the wound and the edge regions provided with the retaining element, is then arranged over the wound and the retaining element such that reversible wound closure takes place through positive engagement of hook-like elements of the retaining element in meshes of the cover element.

Regarding preferred methods, reference is expressly made to the preceding description of the wound closure device itself

Our devices are associated with a whole number of advantages. Thus, a wound closure device is made available with which not only uncomplicated wounds and wound profiles can be quickly and flexibly treated, but also more complicated wounds and wound profiles. We overcame the previous limitation of known wound closure devices to either straight wound profiles requiring relatively simple treatment or more complicated wound profiles requiring complex treatment. Our wound treatment principle is always the same regardless of which type of wound or wound profile is being treated. The user, i.e., generally the physician or operating surgeon, always has the same relatively easy-to-use components of the wound closure device and is able to use these in a familiar manner.

The components of the wound closure device are readily available or are easy to produce. These components involve conventional hook-and loop bands which can preferably be made available with relatively small widths and heights. The wound cover is preferably a simple woven fabric, in particular tulle, which is likewise available at low cost. The materials from which the components of the wound closure device are made, i.e., generally the plastic materials, can largely be chosen freely and adapted to use in medicine. By virtue of using plastic materials, it is relatively easy to ensure a high standard of hygiene. Any necessary modifications can easily be made, for example, either by using an additional film to cover the wound or by connecting the cover element beforehand to a film or coating.

As a result of the retaining mechanism not engaging directly in the wound but being located in proximity to the wound edges, the wound-healing process is not impaired by the wound closure device. This considerably reduces the risk of infection and helps prevent scar formation. This point should be particularly emphasized, since esthetic aspects play an increasingly important role in the treatment of wounds. It should also be noted that the wound closure device particularly facilitates the treatment of wounds in sensitive patients and in children.

Further features will become clear from the following description of preferred examples. The individual features can each be realized singly, or severally in combination with one another. The described examples serve only for explanatory purposes and for better understanding and are not in any way to be interpreted as limiting.

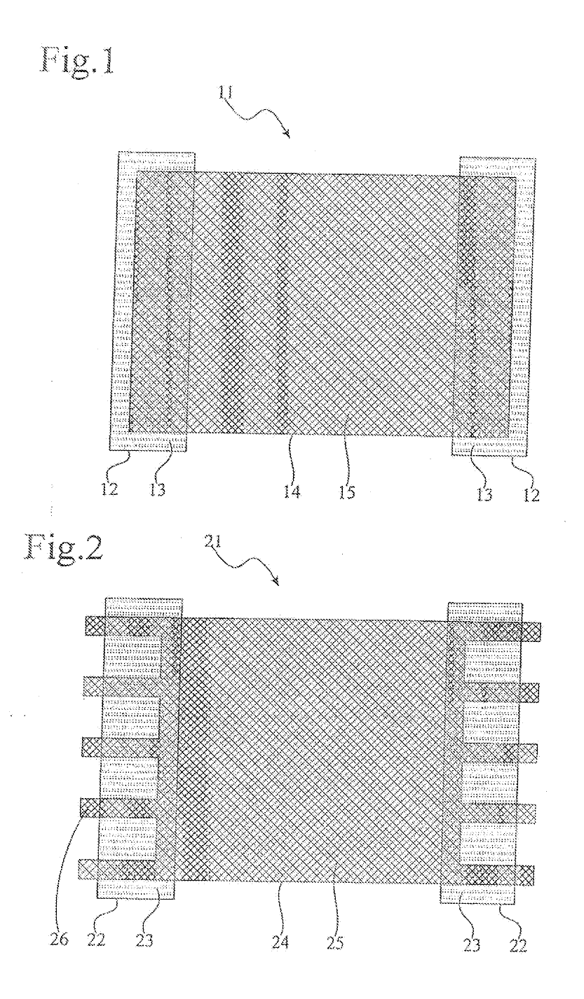

FIG. 1 shows the wound closure device 11 in a schematic plan view. This wound closure device 11 consists of two band-like or strip-like retaining elements 12 which, on their top face directed toward the observer, have a multiplicity of hook-like elements 13. On their faces directed away from the observer, the retaining elements 12 each have an adhesive layer, which adhesive layers (not shown in FIG. 1) are provided for securing the retaining elements 12 to the tissue surrounding the wound.

Finally, FIG. 1 shows the gauze-like cover element 14 as a component part of the wound closure device 11. This cover element 14 consists of a fabric with a multiplicity of meshes 15, which are indicated only symbolically in FIG. 1. These meshes 15 are provided to cooperate with the hook-like elements 13 of the retaining elements 12 to effect closure of the wound.

At this point, it will be noted for all the drawings that the chosen views are idealized. This also applies in particular to the views of the gauze-like cover elements (see, for example, cover element 14 according to FIG. 1). For example, it is clear to those skilled in the art that the meshes shown in the drawings for the cover elements (see, for example, meshes 15 of the cover element 14) are not uniform across the entire cover element, and instead have a different size depending on the tension applied to the wound for closing the latter, which tension can in turn be of a different order in different directions. For reasons of clarity, this has not been shown in the drawings.

As FIG. 1 shows, a wound present between the retaining elements 12 is completely covered by the cover element 14. This coverage also comprises the edge regions of the wound and a part of the surface of the retaining elements 12.

FIG. 2 shows another example 21 of the wound closure device. Here too, two band-like or strip-like retaining elements 22 are provided which, on their top face directed toward the observer, have a multiplicity of hook-like elements 23. Here too, an adhesive layer (not shown) for securing the retaining elements 22 to the tissue surrounding the wound is provided on the undersides of the retaining elements 22.

The wound closure device 21 according to FIG. 2 also comprises the gauze-like cover element 24 which consists of a fabric with meshes 25. This cover element 24 according to FIG. 2 is designed such that, on two of its opposite edges, it has narrow surface regions 26 provided to orient the cover element 24 with respect to the wound and with respect to the retaining elements 22. These surface regions can also be regarded as continuations 26 of the cover element 24. As has already been explained in the description, these surface regions (also in the sense of tensioning means) can serve to stretch the cover element 24 across the wound and then secure it to the retaining elements 22. The surface regions 26 also make it easier to pull the cover element 24 off from the retaining elements 22 if the wound region is to be opened again and made accessible from above. In this connection, it can be advantageous if the surface regions 26 extend beyond the retaining elements 22, as is shown in FIG. 2. In this way, the surface regions 26 can be easily gripped and handled by the user.

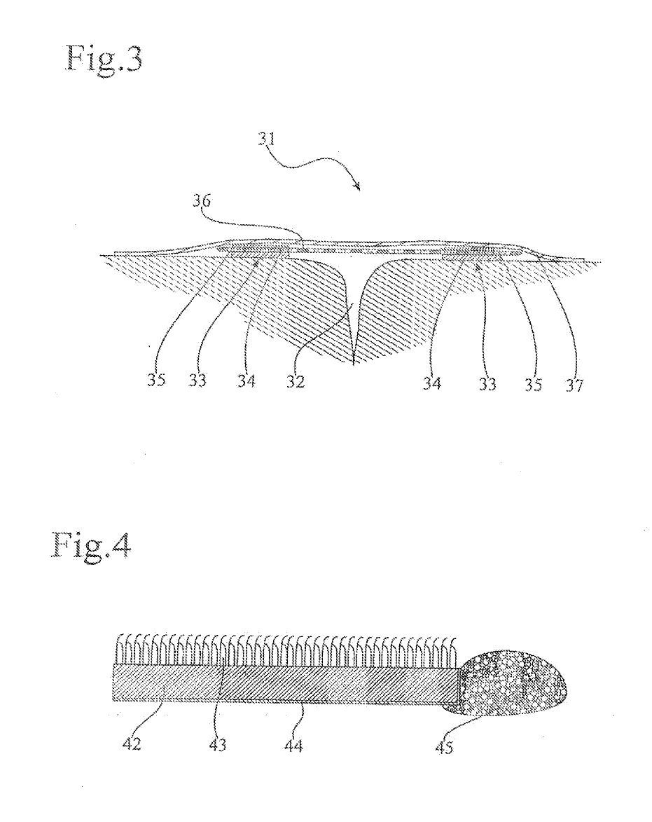

FIG. 3 shows a wound closure device 31 applied over a wound and additionally provided with a film (affixed at the edges) covering the wound closure device. This arrangement is shown in FIG. 3 in a schematic cross section.

FIG. 3 shows the wound 32 with the tissue region surrounding it.

To both sides of the wound 32, band-like or strip-like retaining elements 33 are applied which, in the case of FIG. 3, extend perpendicular to the plane of the drawing. On their underside, these retaining elements 33 have adhesive layers 34, with the aid of which they are secured (reversibly) to the tissue. On their top faces, the retaining elements 33 have a multiplicity of hook-like elements 35.

FIG. 3 also shows the gauze-like cover element 36 which is formed from a fabric and has a multiplicity of meshes (not shown in detail in FIG. 3). According to FIG. 3, this cover element 36 is arranged such that the wound 32 can be closed by a positive engagement of at least some of the hook-like elements 35 of the retaining elements 33 in the meshes of the cover element 36. This closing is generally achieved by the cover element 36 being connected to one of the two retaining elements and by the two edges of the wound 32 then being approximated (adapted) to each other by tension. The cover element 36 is then also connected to the second retaining element 33. The tensioning of the wound edges relative to each other can be temporarily supported by external aids.

FIG. 3 finally shows another film 37 arranged above the wound closure device 31 composed of retaining elements 33 and cover element 36. As has been explained in the description, this film 37 serves to cover the wound completely (in a germ-proof manner) from the environment. As has already been mentioned too, this film can be permeable to gas and/or permeable to water vapor, for example, made of polyurethane to ensure exchange of substances between wound and environment. The film 37 is affixed at its edge regions to the tissue surrounding the wound.

FIG. 4 shows a particular example of a band-like or strip-like retaining element that can be used. This retaining element 42 is used to reduce or prevent the movement of wound liquid, blood or the like from the wound into the hook structure of the retaining element. For this purpose, in addition to the hook-like elements 43 on its top face and possibly in addition to an adhesive layer 44 on its underside, the retaining element also has, on at least one of its two (longer) edges, a liquid-retaining and liquid-repelling means in the form of a bead-like foam strip 45. This foam strip 45, which can also be provided on both edges of the retaining element 42, is preferably made of polyethylene. The foam strip 45 is preferably affixed to the corresponding edge region of the retaining element 42 and/or in the area of the underside of the retaining element 42. When used as intended for closing a wound, the retaining element 42 is secured on the edge of the wound in such a way that the foam strip 45 is directed toward the wound edge.

In this case, the foam strip 45 performs two functions. On the one hand, by virtue of its design as a bead, it keeps the wound liquid away from the retaining element 42 and therefore from the hook-like elements 43. It therefore performs a sealing function. On the other hand, by virtue of the material chosen (foam), the strip 45 is able to take up wound liquid, such that the aforementioned sealing function is supported. All of this prevents wound liquid from penetrating into the hook structure of the retaining element 42. In this way, the hygiene problems associated with the wound-healing process and already discussed in the description are prevented or at least reduced.

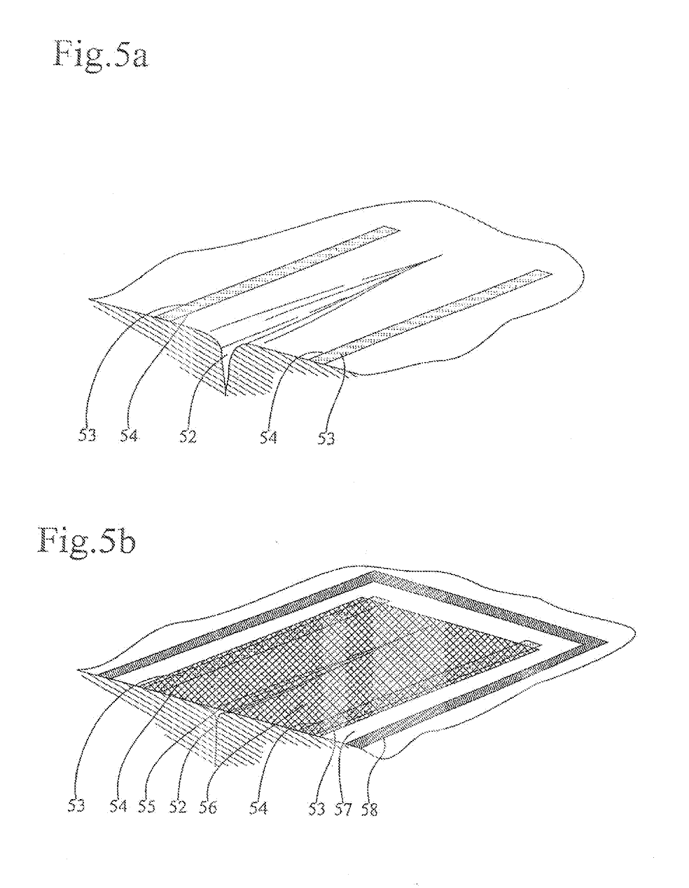

Finally, FIG. 5 shows a wound being closed in the way made possible with the aid of the wound closure device.

For this purpose, in a first stage, a retaining element 53 is applied to both sides of the wound 52 which, in the case shown, has an elongate and curved profile. The schematic view in FIG. 5 shows that the retaining elements 53 are oriented parallel to each other and in a straight line. The tensioning of the wound, which is not uniform across the wound profile, is then compensated by the irregular mesh width in the cover element 55. On their top face directed toward the observer, the retaining elements 53 have the hook-like elements 54 already described.

This first method stage is illustrated in FIG. 5a.

FIG. 5b shows a later stage in the closure of the wound 52, at which stage the wound closure is substantially complete. The wound is substantially closed, and, by virtue of the chosen arrangement, the meshes 56 of the gauze-like cover element 55 are in positive engagement with the hook-like elements 54, such that the edges of the wound 52 remain tensioned with respect to each other.

As has already been explained, the wound closure can be effected by the cover element 55 first of all being secured on one side to one retaining element 53 and by the wound then being closed with tensioning. In doing this, the meshes 56 of the gauze-like cover element 55 are stretched and widened, the elasticity needed for this being made available by the thread material used for the configuration of the mesh and/or by the structure of the mesh. The cover element 55 is then connected to the retaining element 53 on the other side. This can be undertaken or supported by external aids that the user briefly uses to draw the wound together.

To protect the wound from external contamination (water, particles, germs) and provide moisture control, a gas-permeable film 57 is affixed at its edge regions 58 across the cover element 55, which film 57 can be removed without in so doing loosening the cover element 55.

FIG. 5b also shows that, by virtue of the mesh-like structure of the cover element, the wound remains at least partially visible from above, such that the wound-healing process can be monitored. Depending on the wound profile, the wound closure device can be opened at any time and, if appropriate, closed again, such that an adjustable and adaptable wound closure device is afforded.

1. An adjustable wound closure device comprising:
at least one band or strip retaining element that attaches in a region of at least one edge of a wound, wherein the retaining element has a multiplicity of hook elements on at least part of at least one of its two faces; and
at least one gauze cover element that substantially completely covers the wound and the edge regions provided with the retaining element,
wherein the hook elements of the retaining element and the meshes of the cover element arranged such that the wound can be reversibly closed by a positive engagement of at least some of the hook elements in the meshes.

2. The wound closure device as claimed in claim 1, further comprising an additional film arranged over the wound closure device after the wound has been closed, and the film is optionally provided with adhesives in edge regions optionally protruding beyond the wound closure device.

3. The wound closure device as claimed in claim 2, wherein the film is permeable to water vapor.

4. The wound closure device as claimed in claim 2, wherein the film is a plastic film comprising polyurethane.

5. The wound closure device as claimed in claim 1, wherein the retaining element comprises a biocompatible plastic.

6. The wound closure device as claimed in claim 1, wherein the retaining element on a face directed away from the hook-like elements has adhesive to apply the retaining element to the wound edge.

7. The wound closure device as claimed in claim 1, wherein the retaining element has a width of less than 20 mm.

8. The wound closure device as claimed in claim 1, wherein the retaining element, including the hook elements, has a height of 0.5 mm to 5 mm.

9. The wound closure device as claimed in claim 1, wherein the hook elements of the retaining element are in the shape of a mushroom head.

10. The wound closure device as claimed in claim 1, wherein the hook elements of the retaining element have harpoon-shaped barbs.

11. The wound closure device as claimed in claim 1, wherein the gauze cover element is a woven tulle fabric.

12. The wound closure device as claimed in claim 1, wherein the gauze cover element comprises non-resorbable plastic threads.

13. The wound closure device as claimed in claim 1, wherein the gauze cover element has a weight of 10 to 100 g/m².

14. The wound closure device as claimed in claim 1, wherein the gauze cover element has an elongation at break of 30% to 100%.

15. The wound closure device as claimed in claim 1, wherein, on at least one of the edges of the gauze cover element, at least one planar means, or a means formed from individual threads or a plurality of threads, is provided for orienting the cover element with respect to the wound and to the retaining element.

16. The wound closure device as claimed in claim 1, wherein the gauze cover element, on at least one of its two faces, is provided at least partially with a coating or film.

17. The wound closure device as claimed in claim 16, wherein the coating or film is permeable to water vapor.

18. The wound closure device as claimed in claim 16, wherein the coating or film comprises a coating or film of polyurethane.

19. The wound closure device as claimed in claim 1, wherein the mesh width of the gauze cover element is at least twice as great as the diameter of the hook-like elements of the retaining element.

20. The wound closure device as claimed in claim 19, wherein the mesh width of the gauze cover element is two to four times as great as the diameter of the hook elements of the retaining element.

21. The wound closure device as claimed in claim 1, wherein an arrangement of the hook elements on the retaining element is chosen such that between 1 and 3 hook elements fit into one mesh of the gauze cover element.

22. The wound closure device as claimed in claim 21, wherein, relative to the arrangement of the retaining element on the wound edge, different numbers of hook elements of the retaining element fit into one mesh in a direction of the wound profile and in a direction of wound tensioning to be applied to the wound of the gauze cover element, with one hook element fitting into one mesh in a horizontal direction and two hook elements fitting into one mesh in a vertical direction.

23. The wound closure device as claimed in claim 1, wherein the holding strength of the wound closure device on the wound, upon tensile loading of the gauze cover element perpendicular to the wound profile, is at least 10 N.

24. The wound closure device as claimed in claim 1, wherein the retaining element on at least one of its two longitudinally extending edges has a liquid retainer and/or liquid repellent that can reduce or prevent movement of wound liquid of the wound into the hook structure of the retaining element.

25. The wound closure device as claimed in claim 24, wherein the liquid retainer/repellent is a layer or a coating in the form of an edge bead.

26. The wound closure device as claimed in claim 25, wherein the layer or coating comprises polyurethane or polyethylene.

27. The wound closure device as claimed in claim 24, wherein the retainer/repellent is a foam.

28. The wound closure device as claimed in claim 1, wherein the wound closure device is in a sterile form arranged in a sterile package.

29. A kit for closing wounds comprising at least one wound closure device as claimed in claim 1 in sterilized form in a sterile package.

30. (canceled)

31. A method for closing wounds, wherein at least one band or strip retaining element, which has a multiplicity of hook-like elements on at least part of at least one of its two faces, is applied in a region of at least one edge of a wound, and at least one gauze cover element, that substantially completely covers the wound and the edge regions provided with the retaining element is arranged over the wound and the retaining element such that wound closure takes place through positive engagement of hook elements of the retaining element in meshes of the cover element.

32. (canceled)
